(19) United States
(12) Patent Application Publication
(10) Pub. No.: US 2005/0020554 A1
(43) Pub. Date: Jan. 27, 2005

(54) Stability of hormone formulations

(76) Inventors: Salah U. Ahmed, New City, NY (US); Sanjeev K. Gupta, Washington Twp., NJ (US); Quamrul H. Majumder, Pomona, NY (US); IItifat Hasan, Boonton, NJ (US)

Correspondence Address: STERNE, KESSLER, GOLDSTEIN & FOX PLLC, 1100 NEW YORK AVENUE, N.W., WASHINGTON, DC, 20005, US

(21) Appl. No.: 10/860,595

(22) Filed: Jun. 4, 2004

Related U.S. Application Data
(60) Provisional application No. 60/476,223, filed on Jun. 6, 2003.

(51) Int. Cl. A61K 31/56

(52) U.S. Cl. 514/182

(57) ABSTRACT

The present invention is directed to a method of preparing a pharmaceutical dosage form, the method comprising (a) encasing a pharmaceutical dosage form comprising an estrogen in a container essentially impervious to oxygen, and (b) purging the container with an inert gas, and pharmaceutical dosages formed by the method thereof. The present invention is also directed to a pharmaceutical kit comprising (a) a container impervious to oxygen, wherein the container contains an atmosphere of essentially inert gas, and (b) a pharmaceutical dosage form, wherein the pharmaceutical dosage form comprises an estrogen and wherein the pharmaceutical dosage form is encased in the container.

This application claims priority to U.S. Provisional Patent Application 60/476,223, filed Jun. 6, 2003, which is incorporated herein in its entirety.

### BACKGROUND OF THE INVENTION

1. Field of the Invention

The present invention is directed to a method of preparing a pharmaceutical dosage form, the method comprising (a) encasing a pharmaceutical dosage form comprising an estrogen in a container essentially impervious to oxygen, and (b) purging the container with an inert gas, and pharmaceutical dosage forms made by the method thereof. The present invention is also directed to a pharmaceutical kit comprising (a) a container impervious to oxygen, wherein the container contains an atmosphere of essentially inert gas, and (b) a pharmaceutical dosage form comprising an estrogen, wherein the pharmaceutical dosage form is encased in the container.

2. Background

Estrogen formulations are used for various purposes. For example, estrogen formulations have been employed for replacement therapy of estrogen deficiency associated with menopausal symptoms, female hypogonadism, amenorrhea, female castration and primary ovarian failure. Additionally, estrogens are widely used in contraceptive formulations.

Many estrogen formulations are unstable and over time degrade from an active form to a less active or inactive form. Estrogenic degradation presents problems associated with storage of pharmaceutical dosage forms containing estrogens. Likewise, the degradation over time of estrogenic formulations creates difficulty in assessing the actual dosage amount of active estrogen being administered.

Estrogenic degradation in an estrogen formulation can be reduced by environmental factors. Buffering agents capable of maintaining the pH of an aqueous estrogen solution between 6.5 to 7.5 can be used (U.S. Pat. No. 2,834,712). Antioxidants can be used to stabilize synthetic conjugated estrogens (U.S. Pat. No. 4,154,820).

A need exists in the art for improved methods to stabilize pharmaceutical dosage forms comprising estrogens. Stabilized estrogens in pharmaceutical dosage forms would allow for longer storage periods, and would allow the amount of estrogenic components to remain constant over the storage period.

### BRIEF SUMMARY OF THE INVENTION

It is an object of the invention to provide a method of preparing a pharmaceutical dosage form, the method comprising (a) encasing a pharmaceutical dosage form comprising an estrogen in a container essentially impervious to oxygen, and (b) purging the container with an inert gas.

It is an object of the invention to provide a pharmaceutical kit, the pharmaceutical kit comprising (a) a container impervious to oxygen, wherein the container contains an atmosphere of essentially inert gas, and (b) a pharmaceutical dosage form comprising an estrogen, wherein the pharmaceutical dosage form is encased in the container.

The present invention is also directed to the pharmaceutical dosage forms made by the method of the invention as described herein, and uses thereof.

### DETAILED DESCRIPTION OF THE INVENTION

The present invention provides a method of preparing a pharmaceutical dosage form, the method comprising (a) encasing a pharmaceutical dosage form comprising an estrogen in a container essentially impervious to oxygen, and (b) purging the container with an inert gas.

The present invention provides a pharmaceutical kit comprising (a) a container impervious to oxygen, wherein the container contains an atmosphere of essentially inert gas, and (b) a pharmaceutical dosage form comprising an estrogen, wherein the pharmaceutical dosage form is encased in the container.

The present invention also provides a pharmaceutical dosage form made by the method of the invention as described herein, and uses thereof.

The present invention further provides a method of reducing the environmental effects on the estrogenic components of a pharmaceutical dosage form, by purging the container holding the dosage form with an inert gas, thereby reducing the moisture content and oxygen present in the storage container. Purging is the process of essentially evacuating or ridding the container cavity of environmental gases (e.g., oxygen and vaporized water) by introducing an inert gas into the container cavity. Purging with an inert gas allows the pharmaceutical dosage form to be stored in an environment of reduced moisture and oxygen inside the container.

In the present invention, the container is essentially impervious to oxygen gas. Essentially impervious to oxygen gas is defined as essentially not allowing passage of oxygen gas into the inside of the container, where the pharmaceutical dosage form resides. Thus, a container essentially impervious to oxygen would provide a container whose interior, upon removal of oxygen gas, would essentially not allow passage of oxygen gas back into the interior.

Materials capable of forming a container essentially impervious to gas can be used, and are known to those in the art. For example, suitable materials capable of forming a container essentially impervious to oxygen gas include, but are not limited to, single or multiple layers of polyvinyl chloride (PVC), polyvinylidene chloride (PVDC), polychlorotrifluoroethylene (PCTFE), polyethylene (PE), (chloro)tri-fluoro ethylene (CTFE), cyclic polyolefins (CPO), cyclic olefins copolymers (COC), and combinations thereof. In some embodiments, the container can be made of a bilayer or multiple layers of, e.g., PVC and PVDC.

In the present invention, the pharmaceutical dosage form is stored in an inert gas. In some embodiments, the pharmaceutical dosage form can be stored in an inert gas for an extended period of time. In some embodiments, the pharmaceutical dosage form is stored in an atmosphere of the inert gas between about 0 months to about 6 months. In some embodiments, the pharmaceutical dosage form is stored in an atmosphere of the inert gas between about 3 months to about 6 months. In some embodiments, the pharmaceutical dosage form is stored in an atmosphere of the inert gas between about 3 months to about 1 year. In some embodiments, the pharmaceutical dosage form is stored in an atmosphere of the inert gas between about 6 months to about 1 year. In some embodiments, the pharmaceutical dosage form is stored in an atmosphere of the inert gas for about 3 months or greater. In some embodiments, the pharmaceutical dosage form is stored in an atmosphere of the inert gas for about 6 months or greater. In some embodiments, the pharmaceutical dosage form is stored in an atmosphere of the inert gas for about 1 year to about 2 years, or greater.

The container of the present invention can encase a various number of dosage forms. In some embodiments, the container encases multiple dosage forms. In some embodiments, the container encases a single dosage form. In some embodiments, the container is a blister pack.

The present invention is directed to a pharmaceutical dosage form comprising estrogen. In some embodiments, the pharmaceutical dosage form is a contraceptive, such as an oral contraceptive. In some embodiments, the pharmaceutical dosage form is useful for hormone replacement therapy. In some embodiments the pharmaceutical dosage form is a solid (e.g., tablet, capsule or caplet).

The pharmaceutical dosage form of the present invention comprises a hormone, such as, but not limited to, a hormone sensitive to oxygen (i.e., having its stability susceptible to the presence of oxygen). In some embodiments, the hormone is an estrogen. In some embodiments, the estrogen is a natural estrogen. In some embodiments, the estrogen is a synthetic estrogen. Estrogens include, but are not limited to, estradiol, estradiol-17β, estradiol valerate, conjugated equine estrogens, piperazine estrone sulphate, estrone, estriol, estriol succinate, polyestriol phosphate, ethinyl estradiol, quinestranol, mestranol and combinations thereof. In some embodiments, the estrogen is ethinyl estradiol. In some embodiments, the estrogen is sensitive to oxygen.

The pharmaceutical dosage form of the present invention can further comprise a progestin. Progestins include, but are not limited to, dl-norgestrel, levonorgestrel, norethindrone (norethisterone), norethindrone acetate, ethynodiol diacetate, medroxyprogesterone acetate, cyproterone acetate, trimegestone, norethynodrel and combinations thereof. In some embodiments, the progestin is levonorgestrel.

In the present invention, various inert gases can be used. An inert gas is an element or molecule in the form of a gas that is either completely, substantially or essentially unreactive. Inert gases include, but are not limited to, nitrogen, argon, carbon dioxide, helium, neon, krypton and xenon. In some embodiments, the inert gas is nitrogen.

In the present invention, various concentrations of the inert gas can be contained inside of the container. In some embodiments, the inert gas in the container is between about 80% to about 100% by volume of the total gaseous content. In some embodiments, the inert gas in the container is between about 90% to about 100% by volume of the total gaseous content. In some embodiments, the inert gas in the container is between about 95% to about 100% by volume of the total gaseous content. In some embodiments, the inert gas in the container is between about 98% to about 100% by volume of the total gaseous content. Per cent volume of the gaseous content can be determined at one atmosphere pressure and room temperature.

When stored over time, the pharmaceutical dosage form of the present invention prevents or decreases degradation of estrogenic potency compared to pharmaceutical dosage forms that are not prepared according to the method of the present invention. In some embodiments, degradation of the pharmaceutical dosage form of the invention stored for 3 months in the inert gas does not exceed about 5%. In some embodiments, degradation of the pharmaceutical dosage form of the invention stored for 6 months in the inert gas does not exceed about 8%. Degradation can be determined by comparing the amount of estrogen in a tablet prior to the storage period versus the amount of estrogen in a tablet after the storage period.

The present invention can comprise additional stabilizing components.

In some embodiments, the container can contain a desiccant. In some embodiments, the container is enclosed in a pouch. In some embodiments, the pouch is substantially or essentially impervious to moisture. A pouch is known to those in the art, and can include any flexible packaging that encompasses that container. The pouch can further contain a desiccant. A desiccant is any drying agent that removes moisture from the air. Desiccants include, but are not limited to, silica gel, clay desiccants, calcium sulfate, calcium chloride, calcium oxide, zeolite, activated alumina, activated charcoal and combinations thereof. In some embodiments, the desiccant is silica gel. In some embodiments, the pharmaceutical dosage form of the invention can further comprise an antioxidant. In some embodiments, the container and/or the pouch can contain an antioxidant. Antioxidants include, but are not limited to, sodium sulfite, potassium sulfite, metabisulfite, bisulfites, thiosulfates, thioglycerol, thiosorbitol, cysteine hydrochloride, α-tocopherol, and combinations thereof. In some embodiments, the container and/or pouch can contain an oxygen scavenger. Oxygen scavengers include, but are not limited to, ferrous oxide, tocopherol pellets, sodium sulfite, hydrazine hydrate, N-N-diethyl hydroxlamine (DEHA), morpholine, cyclohexamine, diethyl amino ethanol, and combinations thereof.

All of the various embodiments or options described herein can be combined in any and all variations.

The following example serves only to illustrate the invention, and is not to be construed in any way to limit the invention.

### EXAMPLE

Blister packets were created using an Uhlmann UPS-4 blister packet machine. A tablet containing 0.03 mg ethinyl estradiol and 0.15 mg levonorgestrel was placed in a blister pack film cavity comprising PVC/Aclar® RX 160 (25.4 μm) (Honeywell, Morristown, N.J.). The blister pack cavity containing the tablet was purged with nitrogen gas (18-20 standard cubit feet hour) for greater than 2 seconds. The rate of blistering was greater than 300 blisters/minute. The blister pack was then sealed with Klöckner® foil blister lidding (Klöckner Capital Corporation, Gordonville, Va.). As a control, pharmaceutical dosage forms from the same tablet lot, but not nitrogen purged, were also placed in blister packs. The nitrogen purged blister packs, as well as the control blister packs, were placed in a Pharma Center Shelbyville 92036 pouch (Alcan, Inc., Montreal, Canada).

The blister packs were then stored for either 3 or 6 months. After storage, estrogenic activity was assayed using typical standard analytical methods (U.S. Pharmacopeia, p. 639, United States Pharmacopeial Convention, Inc., Rockville, Md. (1995)). Tablets stored in blister packs purged with nitrogen demonstrated 1.3% degradation of estrogen at 3 months of storage and 1.2% degradation after 6 months of storage. Tablets without nitrogen purging demonstrated 7.4% degradation of estrogen after 3 months of storage, and 8.9% degradation of estrogen after 6 months of storage.

This example illustrates one possible formulation of the present invention. While the invention has been particularly shown and described with reference to some embodiments thereof, it will be understood by those skilled in the art that they have been presented by way of example only, and not limitation, and various changes in form and details can be made therein without departing from the spirit and scope of the invention. Thus, the breadth and scope of the present invention should not be limited by any of the above-described exemplary embodiments, but should be defined only in accordance with the following claims and their equivalents.

All documents cited herein, including journal articles or abstracts, published or corresponding U.S. or foreign patent applications, issued or foreign patents, or any other documents, are each entirely incorporated by reference herein, including all data, tables, figures, and text presented in the cited documents.

1. A method of preparing a pharmaceutical dosage form, said method comprising:
(a) encasing a pharmaceutical dosage form comprising an estrogen in a container essentially impervious to oxygen; and
(b) purging said container with an inert gas.

2. The method of claim 1, wherein said pharmaceutical dosage form is stored in said inert gas.

3. The method of claim 1, wherein said pharmaceutical dosage form is a contraceptive.

4. The method of claim 1, wherein said pharmaceutical dosage form is useful for hormone replacement therapy.

5. The method of claim 1, wherein said container encases a single dosage form.

6. The method of claim 1, wherein said container is a blister pack.

7. The method of claim 1, wherein said estrogen is selected from the group consisting of estradiol, estradiol-17β, estradiol valerate, conjugated equine estrogens, piperazine estrone sulphate, estrone, estriol, estriol succinate, polyestriol phosphate, ethinyl estradiol, quinestranol, mestranol and combinations thereof.

8. The method of claim 1, wherein said estrogen is ethinyl estradiol.

9. The method of claim 1, wherein said pharmaceutical dosage form further comprises a progestin.

10. The method of claim 9, wherein said progestin is selected from the group consisting of dl-norgestrel, levonorgestrel, norethindrone (norethisterone), norethindrone acetate, ethynodiol diacetate, medroxyprogesterone acetate, cyproterone acetate, trimegestone, norethynodrel and combinations thereof.

11. The method of claim 9, wherein said progestin is levonorgestrel.

12. The method of claim 1, wherein said inert gas is nitrogen.

13. The method of claim 1, wherein said inert gas in said container is between about 80% to about 100% by volume of the total gaseous content.

14. The method of claim 1, wherein said inert gas in said container is between about 90% to about 100% by volume of the total gaseous content.

15. The method of claim 1, wherein said inert gas in said container is between about 95% to about 100% by volume of the total gaseous content.

16. The method of claim 1, wherein said inert gas in said container is between about 98% to about 100% by volume of the total gaseous content.

17. The method of claim 1, further comprising enclosing said container in a pouch comprising a desiccant.

18. The method of claim 17, wherein said desiccant is silica gel.

19. The method of claim 1, wherein said pharmaceutical dosage form comprises ethinyl estradiol and levonorgestrel; and
said inert gas is nitrogen gas.

20. A pharmaceutical dosage form made by the method of claim 1.

21. A pharmaceutical kit comprising:
(a) a container impervious to oxygen, wherein said container contains an atmosphere of essentially inert gas; and
(b) a pharmaceutical dosage form comprising an estrogen, wherein said pharmaceutical dosage form is encased in said container.

22. The pharmaceutical kit of claim 21, wherein said pharmaceutical dosage form is a contraceptive.

23. The pharmaceutical kit of claim 21, wherein said pharmaceutical dosage form is useful for hormone replacement therapy.

24. The pharmaceutical kit of claim 21, wherein said container encases a single dosage form.

25. The pharmaceutical kit of claim 21, wherein said container is a blister pack.

26. The pharmaceutical kit of claim 21, wherein said estrogen is selected from the group consisting of estradiol, estradiol-17β, estradiol valerate, conjugated equine estrogens, piperazine estrone sulphate, estrone, estriol, estriol succinate, polyestriol phosphate, ethinyl estradiol, quinestranol, mestranol and combinations thereof.

27. The pharmaceutical kit of claim 21, wherein said estrogen is ethinyl estradiol.

28. The pharmaceutical kit of claim 21, wherein said pharmaceutical dosage form further comprises a progestin.

29. The pharmaceutical kit of claim 28, wherein said progestin is selected from the group consisting of dl-norgestrel, levonorgestrel, norethindrone (norethisterone), norethindrone acetate, ethynodiol diacetate, medroxyprogesterone acetate, cyproterone acetate, norethynodrel and combinations thereof.

30. The pharmaceutical kit of claim 28, wherein said progestin is levonorgestrel.

31. The pharmaceutical kit of claim 21, wherein said inert gas is nitrogen.

32. The pharmaceutical kit of claim 21, wherein said inert gas in said container is between about 80% to about 100% by volume of total gaseous content.

33. The pharmaceutical kit of claim 21, wherein said inert gas in said container is between about 90% to about 100% by volume of total gaseous content.

34. The pharmaceutical kit of claim 21, wherein said inert gas in said container is between about 95% to about 100% by volume of total gaseous content.

35. The pharmaceutical kit of claim 21, wherein said inert gas in said container is between about 98% to about 100% by volume of total gaseous content.

36. The pharmaceutical kit of claim 21, further comprising enclosing said container in a pouch comprising a desiccant.

37. The method of claim 36, wherein said desiccant is silica gel.

38. The pharmaceutical kit of claim 21, wherein
said pharmaceutical dosage form comprises ethinyl estradiol and levonorgestrel; and
said inert gas is nitrogen gas.
